(19) United States
(12) Patent Application Publication
(10) Pub. No.: US 2022/0168206 A1
(43) Pub. Date: Jun. 2, 2022

(54) ACTIVE AGENT FOR USE IN HAIR GROWTH REGULATION

(71) Applicant: MONASTERIUM LABORATORY SKIN & HAIR RESEARCH SOLUTIONS GMBH, Münster (DE)

(72) Inventors: Jérémy Patrick Jean Cherét, Miami, FL (US); Ralf Paus, Hamburg (DE); Thomas Rouille, Münster (DE)

(21) Appl. No.: 17/597,646

(22) PCT Filed: Jul. 14, 2020
(86) PCT No.: PCT/EP2020/069902

§ 371 Date: Jan. 14, 2022

(30) Foreign Application Priority Data
Jul. 17, 2019 (EP) 19186785.2

(51) Int. Cl. A61K 8/60; A61Q 7/00; A61K 8/44

(52) U.S. Cl. CPC A61K 8/606; A61Q 7/00; A61K 31/7105; A61K 8/44; A61K 8/602

(57) ABSTRACT

An active agent for use in hair growth regulation, particularly for use in the treatment of hair growth (stimulation or inhibition), wherein the active agent activates, enhances, inactivates, blocks or dampens the cellular response of the taste receptor TAS2R4 or interferes with the expression of the receptor. Furthermore, the present invention is directed to compositions for use as a cosmetic or medicament in the treatment of hair growth the composition comprising at least one of the aforementioned active agents and at least one auxiliary agent. In addition, a non-therapeutic method of hair growth regulation is disclosed, wherein an effective amount of at least one of the aforementioned active agents is administered to a subject.

### CROSS-REFERENCE TO RELATED APPLICATIONS

This application is a national phase entry under 35 U.S.C. § 371 of International Patent Application PCT/EP2020/069902, filed Jul. 14, 2020, designating the United States of America and published as International Patent Publication WO 2021/009177 A1 on Jan. 21, 2021, which claims the benefit under Article 8 of the Patent Cooperation Treaty to European Patent Application Serial No. 19186785.2, filed Jul. 17, 2019.

### TECHNICAL FIELD

The present disclosure is directed to an active agent for use in hair growth regulation, particularly for use in the treatment of hair growth, wherein the active agent modulates the activity of a taste receptor, including but not limited to activation, enhancement, inactivation, blocking or dampening the cellular response of a taste receptor or interfering with the expression of the receptor. Further, the present disclosure is directed to a composition for use as a cosmetic or medicament in the treatment of hair growth the composition comprising at least one active agent that modulates the activity of a taste receptor, including but not limited to activation, enhancement, inactivation, blocking or dampening the cellular response of a taste receptor or interfering with the expression of the receptor. In addition, the present disclosure is directed to a non-therapeutic method of hair growth regulation, wherein an effective amount of at least one active agent that modulates the activity of a taste receptor, including but not limited to activation, enhancement, inactivation, blocking or dampening the cellular response of a taste receptor or interfering with the expression of the receptor, is administered to a subject.

### BACKGROUND

Evolutionary studies suggest the common ancestor to the hair follicle was a simple glandular structure with a function in regulating hydration, potentially to allow independence of the organism from aquatic environments. Specialization of these simple glands resulted in the evolution of hair follicles with their associated sebaceous and, in some cases, apocrine sweat glands.

The pilosebaceous unit (PSU), an anatomical compartmentalization of the hair follicle and surrounding parts, includes the hair follicle, arrector pili muscle, sebaceous gland and, where present, apocrine sweat gland. The hair follicle is an epithelial-mesenchymal-neuroectodermal interaction unit that is the most densely innervated structure of mammalian skin, and in addition, hair follicle possesses its own immune system. As such, the hair follicle is much more than just a hair-producing organ.

The formation of hair follicles occurs once in the lifetime of healthy mammals, with the number of hair follicles determined in utero. Hair follicle morphogenesis is the generation of the whole hair follicle structure from the epidermis and the mesoderm. During postnatal life, the hair follicle undergoes life-long cyclical transformations where it progresses through stages of rapid growth (anagen), regression (catagen) and relative quiescence (telogen) classified by morphological indicators and molecular markers.

There are a number of common and rare diseases associated with the hair follicle cycle, such as non-scarring alopecia, scarring alopecia, chronic and acute hair shedding, hypertrichosis or hirsutism. Further, there are a number of other conditions, where the control of unwanted hair on the human body or the promotion of hair growth on areas of the human body is desired, such as drug-induced hair loss (e.g., chemotherapy), radiation-induced hair loss (e.g., radiotherapy), unwanted hair growth due to drugs such as cyclosporine A or diazoxide.

### BRIEF SUMMARY

There is a continuous need for active agents for actively controlling hair growth in subjects without undesired side effects. It was found in the present disclosure that a taste receptor TAS2R4 was expressed in the hair follicle epithelium (outer root sheath, inner root sheath, and hair matrix) and in the dermal papilla, suggesting it is involved in regulation of hair growth.

Further, it has been found in the present disclosure that inactivation of TAS2R4 in anagen hair follicles using RNAi or a TAS2R4 inverse agonist, such as Nα,Nα-bis(carboxymethyl)-l-lysine (BCML) results in hair growth promotion, delayed catagen promotion, increase in hair follicle proliferation and pigmentation, associated with upregulation of the expression of pro-anagenic growth factor IGF1 and downregulation of pro-catagenic growth factor TGFβ2. Vice versa, activation of TAS2R4 using agonists, such as Rebaudioside A, Rubusoside and Stevioside, results in hair growth inhibition, accelerated anagen-catagen transition, associated with the reduced hair follicle cell proliferation and pigmentation.

In conclusion, it has been found in the present disclosure that the administration of an active agent that activates, enhances, inactivates, blocks or dampens the cellular response of a receptor or interferes with the expression of the taste receptor TAS2R4 is effective in hair growth regulation.

A “taste receptor” originally describes a type of receptor that facilitates the sensation of taste in the oral cavity. Taste receptors are divided into two families, type 1 (sweet, TAS1R2-TAS1R3), and type 2 (bitter, TAS2R1-TAS2R50, and TAS2R60). Combinations of these receptors in dimers or other complexes contribute to different perceptions of taste. The presence of TAS2R4 is not previously known in hair follicle cells.

The mRNA expression of some but not all TAS2Rs, including (TAS2R3, TAS2R4, TAS2R5, TAS2R8, TAS2R9 TAS2R14, TAS2R30, TAS2R42, and TAS2R60) was detected in the RNA collected from full-thickness skin biopsies (Shaw et al., 2018). Expression of bitter taste receptors TAS2R1 and TAS2R38 has been reported in human epidermal keratinocytes (Wolfle et al., 2015). A number of bitter taste receptors have been reported to be expressed in extraoral/nasal tissues, such as but not limited to the reproductive organs, the upper respiratory tract, the gastrointestinal tract, the brain and the immune system (Behrens et Meyerhof, 2018; Luddi et al., 2019; Patel et al., 2018; Tran et al., 2018).

“TAS2R4” is the official gene symbol for “Taste receptor type 2 member 4” and identifies the protein that is encoded by the TAS2R4 gene in humans (NCBI Gene ID: 50832; HGNC: 14911; NCBI mRNA sequence: NM 016944.1; NCBI protein sequence: NP 058640). The protein is a 7-transmembrane receptor protein, member of the G protein-coupled receptor superfamily and functions as a bitter taste receptor. TAS2R4 RNA expression levels are highest in the skin, reproductive organs, brain and endocrine tissues.

According to one alternative of the present disclosure, an active agent that activates, enhances, inactivates, blocks or dampens the cellular response of the taste receptor TAS2R4 is used in the treatment or regulation of hair growth, wherein the active agent is an agonist or antagonist of the taste receptor TAS2R4.

An active agent that activates, enhances, inactivates, blocks or dampens the cellular response of a receptor is either an agonist or antagonist/inverse agonist of the receptor, wherein an “agonist” is a substance that binds to a receptor and activates or enhances the receptor to produce the cellular response. An “antagonist” is a substance that binds to a receptor and blocks or dampens the cellular response to an agonist rather than activating or enhancing it like an agonist. An “inverse agonist” is a substance that binds to a receptor and inactivates the cellular response to an agonist rather than activating or enhancing it like an agonist.

Accordingly, an inventive agonist of the taste receptor TAS2R4 activates or enhances the TAS2R4 receptor to produce the cellular response, whereas the inventive antagonist of the taste receptor TAS2R4 blocks or dampens the TAS2R4 receptor response to endogenous agonists, and the inventive inverse agonist of the taste receptor TAS2R4 inactivates the TAS2R4 receptor response to endogenous agonists, to produce the cellular response.

In those embodiments where the active agent is an agonist of the taste receptor TAS2R4, the active agent is used in the treatment of unwanted hair growth. In those embodiments where the active agent is an antagonist/inverse agonist of the taste receptor TAS2R4, the active agent is used in the treatment of unwanted hair loss.

In specific embodiments of the present disclosure, the taste receptor agonist/antagonist/inverse agonist used is a TAS2R4 agonist selected from the group consisting of amarogentin, arborescin, artemorin, azathioprine, chlorpheniramine, dapsone, D-camphor, denatonium benzoate, diphenidol, dulcoside A, (-)-epicatechin, leu-leu-leu, leu-trp, parthenolide, propylthiouracil, phe-trp, quassin, quinine, rebaudioside A, rebaudioside B, rebaudioside C, rubusoside, steviolbioside, stevioside, sucralose, taurocholic acid, trp-leu, trp-phe, trp-pro, trp-trp, trp-trp-trp, xanthoxin, yohimbine, and combinations thereof. Alternatively, the TAS2R4 agonist is an aptamer binding to the TAS2R4 receptor and activating or enhancing the receptor to produce the cellular response.

The term “aptamer” as used herein refers to DNA, RNA or XNA oligonucleotide or peptide molecules that bind to a specific target molecule, such as receptor molecules.

In other embodiments of the present disclosure, the taste receptor agonist/antagonist/inverse agonist used is a TAS2R4 antagonist/inverse agonist selected from the group consisting of carboxymethyllysine (CML), Nα,Nα-bis(carboxymethyl)-L-lysine hydrate (BCML), glyoxal-derived lysine dimer (GOLD), 4-(2,2,3-trimethylcyclopentyl) butanoic acid (GIV3727), and combinations thereof. Alternatively, the TAS2R4 antagonist/inverse agonist is an aptamer binding to the TAS2R4 receptor and inactivating, blocking or dampening the receptor to produce the cellular response.

According to an alternative of the present disclosure, an active agent that interferes with the expression of the taste receptor TAS2R4 is used in hair growth regulation. An active agent that interferes with the expression of the taste receptor TAS2R4 is either miRNA, siRNA or a ribozyme targeted to the TAS2R4 gene or targeted to the mRNA corresponding to the TAS2R4 gene.

The term “miRNA” refers to microRNA, i.e., small non-coding RNA molecules containing 21 to 23 nucleotides and functioning in RNA knock-down and post-transcriptional regulation of gene expression. miRNAs function via base-pairing with complementary sequences within mRNA molecules. As a result, these mRNA molecules are knocked-down, by cleavage, destabilization and/or less efficient translation of the mRNA.

The term “siRNA” refers to small interfering RNA, i.e., small non-coding RNA molecules, 20 to 25 base pairs in length. siRNA interferes with the expression of specific genes with complementary nucleotide sequences by degrading mRNA after transcription, thereby preventing translation.

According to the present disclosure, a gene is “targeted” by a miRNA, siRNA or a ribozyme when the miRNA, siRNA or a ribozyme molecule selectively decreases or inhibits the expression of TAS2R4. The phrase “selectively decrease or inhibit” as used herein refers to miRNA, siRNA or a ribozyme that affects the expression of TAS2R4.

In specific embodiments of the present disclosure, miRNA or siRNA interfere with the gene expression of the taste receptors TAS1R3 or TAS2R4 by hybridizing under stringent conditions to the gene transcript, i.e., the TAS1R1 or TAS2R4 mRNA, wherein hybridizing “under stringent conditions” means annealing to the target mRNA region, under standard conditions, e.g., high temperature (e.g., <60° C. for 2 hours) and/or low salt content (e.g., 0.1×SSC), which tend to disfavor hybridization.

According to the present disclosure, one of the above-mentioned active agents is used in the treatment of hair growth. In specific embodiments of the present disclosure, the treatment is effected locally, i.e., in, on or at the skin area to be treated. In some embodiments of the present disclosure, the treatment is effected topically, wherein the term “topical” refers to a medication that is applied to a particular place on the skin and/or its appendages, such as hair. In specific embodiments, the topical application is epicutaneous, meaning that the agonist or antagonist is applied directly to the skin. In other embodiments of the present disclosure, the treatment is effected transdermally, wherein the term “transdermal” refers to a medication that is applied across the Stratum corneum into the deeper skin layers.

The term “treatment” as used herein refers to any action resulting in the change of a physical condition. Particularly, the “treatment of hair growth” refers to any change of an initial hair growth condition, such as unwanted presence of hair, unwanted lack of hair, unwanted slow/fast hair growth, chemotherapy-related hair loss, drug-related hair growth and radiation-related hair loss. In other words, the present disclosure is directed to any kind of hair growth regulation.

In specific embodiments of the present disclosure, the above-mentioned active agent is used as a cosmetic in the treatment of hair growth. Particularly, the use as a cosmetic occurs non-therapeutically, but in order to achieve a change of an initial hair growth condition, such as unwanted presence of hair, unwanted lack of hair, unwanted slow/fast hair growth, wherein the initial condition is not caused by a disease or disorder.

In those embodiments where the above mentioned active agent is used as a cosmetic, the active agent used should be cosmetically acceptable, wherein “cosmetically acceptable” means that the active agent should not be toxic or harmful or have any other detrimental side effects upon application on hair and/or skin.

In other specific embodiments of the present disclosure, the above mentioned active agent is used as a medicament in the topical treatment of hair growth disorder, wherein the term “disorder” refers to any functional abnormality or disturbance of the normal healthy condition, and the term “medicament” refers to a substance useful in curing, treating or preventing a condition of disorder.

In some embodiments, the above mentioned active agent is used as a medicament in the topical treatment of a hair growth disorder being selected from the group including nonscarring (noncicatricial) alopecia, such as alopecia areata, telogen effluvium, androgenetic alopecia and anagen effluvium, scarring (cicatricial) alopecia, such as cutaneous lichen planopilaris, discoid lupus erythematosus, dissecting cellulitis and folliculitis decalvans, hypertrichiosis and hirsutism.

In those embodiments where the above mentioned active agent is used as a medicament, the active agent used should be pharmaceutically acceptable, wherein “pharmaceutically acceptable” means that the active agent should not be toxic or harmful or have any other detrimental side effects upon application on hair and/or skin.

In some embodiments, at least one of the inventive active is used as an ingredient of a composition for use as a cosmetic or medicament in the topical treatment of hair growth the composition further comprising at least one auxiliary agent selected from the group including carriers, recipients, adjuvants, diluents, and disintegrants.

In specific embodiments of such compositions, the auxiliary agent is selected from the group including liposomes, nanoparticles, carboxymethyl cellulose, hydroxyethyl cellulose, mineral oil, petrolatum, glycerin, polysorbate 80, hydroxyethyl starch, dextran, and polyethylene glycol.

In the inventive compositions, the concentration of the active agent usually is in the range of from 10 to 10,000 μM. In some embodiments, the lower limit for the concentration of the active agent is 30 μM or even 100 μM. In some embodiments, the upper limit is 3,000 μM or 1,000 μm. This results in preferred ranges of e.g., from 30 to 10,000 μM, from 30 to 3,000 μM, from 10 to 3,000 μM, 100 to 3,000 μM and the like.

In specific embodiments, such compositions further comprise at least one other active agent being effective in the treatment of hair growth.

In such embodiments, the other active agent may be selected from hair growth inhibitors, such as inhibitors of omithine decarboxylase (including Difluoromethylornithine (DFMO)), antiandrogen compounds, inhibitors of 5-α-reductase, inhibitors of androgen receptor, inhibitors of S-adenosyl methionine decarboxylase, inhibitors of 7-glutamyl transpeptidase, inhibitors of adenylosuccinate synthetase, inhibitors of aspartate transcarbamylase, inhibitors of transglutaminase, inhibitors of L-asparagine synthetase, pantothenic acid and its analogues, sulfhydryl reactive compounds, inhibitors of lipoxygenase, inhibitors of cyclooxygenase, inhibitors of nitric oxide synthetase, inhibitors of omithine amino transferase, inhibitors of cysteine synthesis pathway enzymes, inhibitors of protein kinase C, catechin compounds, green tea polyphenols, non-steroidal angiogenesis suppressors, inhibitors of arginase, inhibitors of the metabolic pathway for the conversion of glucose to acetyl-CoA, compounds that inhibit the formation of glycoprotein, proteoglycans, and glycosaminoglycans, inhibitors of matrix metalloproteinase, inhibitors of the cholesterol synthesis pathway, inhibitors of DNA topoisomerase, inhibitors of aminoacyl-tRNA synthetase, inhibitors of the hypusine biosynthesis pathway, compounds that activate androgen conjugation, inhibitors of alkaline phosphatase, inhibitors of protein tyrosine kinase, and compounds that increase cellular ceramide levels. Specific examples include cyproterone acetate, progesterone, acivicin, anthglutin, L-alanosine, guanidino-succinic acid, ethacrynic acid, D-pantothenic acid, pantoyl alcohol, gabaculin, canaline, isonicotinic acid, verapamil, phentolamine, pentosan polysulfate, nafoxidine, tripelennamine, octapine, phloretin, argaric acid, simvastatin, atorvastatin, lovastatin, fluvastatin, mevastatin, NG-methyl-L-arginine, NG-nitro-L-arginine, benzoyl-L-argininamide, L-argininamide, quercetin, apigenin, nordihydroguaratic acid (NDGA), ketoprofen, naproxen, tolmetin, diclofenac, diflunisal, sulindac, thiosalicylic acid, cysteamine, diethyldithiocarbamic acid, D-penicillamine, N-acetyl-L-cysteine, bathocuproine, enalapril, tamoxifen, cimetidine, mycophenolic acid, tetracycline, doxycycline, minocycline, thioridazine, trifluoperizine, 1-(5-isoquinolinylsulfonyl)-2-methylpiperazine, glycyrrhetinic acid, epigallocatechin gallate, epicatechin gallate, epigallocatechin, epicatechin, fusidic acid, and nitroso-acetyl-pencillamine.

In yet other embodiments, the other active agent may be selected from preventatives of chemotherapy- or radiation-induced alopecia or hair loss, such as 4-((cyanoimino((1,2,2-trimethylpropyl)amino)methyl)amino)benzonitrile, epidermal growth factor, fibroblast growth factors, including but not limited to keratinocyte growth factor (FGF7), prostaglandins, cyclin dependent kinases, p53 inhibitors, capase-3 inhibitors, N-acyl cysteine, parathyroid hormone antagonist, alpha-tocopherol, cyclosporine, angiotensin receptor blockers, and minoxidil.

Generally, the inventive composition may be used in any formulation suitable for treatment of hair growth. In specific embodiments of the present disclosure, the composition is formulated in the form of an ointment, a lotion, a cream, a shampoo, a gel, a spray, a plaster or a sustained release plaster. In other specific embodiments of the present disclosure, the composition is formulated in the form of a solution. The solution may be applied by way of a microneedle device or prior to sonication, electrical stimulation, etc.

As mentioned above, the inventive use of the above mentioned active agent may also occur non-therapeutically, wherein non-therapeutically refers to a treatment not being directed to curing, treating or preventing a condition of disorder (see above).

Therefore, the present disclosure is further directed to a non-therapeutic method of hair growth regulation, wherein an effective amount of at least one of the above mentioned active agent is administered to a subject.

The non-therapeutic method also encloses embodiments where the above mentioned active agent is administered to the subject to be treated simultaneously, sequentially or separately together with at least one other active agent useful in the treatment of hair growth (see above).

### BRIEF DESCRIPTION OF THE DRAWINGS

The following examples show some of the features of specific embodiments of the present disclosure. However, the skilled reader will understand that those embodiments are just exemplary but do not restrict the inventive idea to exactly the features or the combination of features of the embodiments of the examples.

In the description of the examples it is referred to the following figures, wherein

FIG. 1 shows the result of an immunofluorescence analysis of TAS2R4 expression in human anagen hair follicle (cf. Example 1).

FIG. 2 shows effect of TAS2R4 knockdown on hair follicle biology, siRNA-targeting TAS2R4 (cf. Example 2) (Mean+/−SEM, n=5 HFs/group, GraphPad Prism 6 (GraphPad, San Diego, Calif.), ***p<0.001, Student's unpaired t-test).

FIG. 3 shows the data of the treatment of human hair follicles with 120 μM Nα,Nα-bis(carboxymethyl)-1-lysine hydrate (BCML) (cf. Example 3) (Mean+/−SEM, n=4-7 HFs/group, GraphPad Prism 6).

FIG. 4 shows the data of the treatment of human hair follicles with 200 μM Rebaudioside A (cf. Example 4) (Mean+/−SEM, n=4-7 HFs/group from 2 donors, GraphPad Prism 6).

FIG. 5 shows the data of the treatment of human hair follicles with 50 μM Rubusoside (cf. Example 5) (Mean+/−SEM, n=6-7 HFs/group, GraphPad Prism 6).

FIG. 6 shows the data of the treatment of human hair follicles with 200 μM Stevioside (cf. Example 6) (Mean+/−SEM, n=5-8 HFs/group, GraphPad Prism 6).

### DETAILED DESCRIPTION

### Examples

1. Immunofluorescence Analysis of TAS2R4 Expression in Human Hair Follicles

Cryosection without fixation stored at −80° C. was dried for 10 min at room temperature (RT) and then fixed in acetone for 20 min at −20° C. Slides were then washed in Tris-buffered saline (TBS) for 5 min at RT (repeat ×2). The sample sections were encircled with a wax marker pen and the slides placed in a humidified chamber. Sample sections were pre-treated with 10% normal goat serum diluted in TBS for 20 min at RT. 1:100 dilution of rabbit polyclonal anti-TAS2R4 (ThermoFisher Cat No.: OSR00153W) in green antibody diluent was added to sample sections and incubated overnight (ON) in a humidified chamber at 4° C. Slides were washed in TBS for 5 min at RT (repeat ×2). 1:1000 dilution of goat anti-rabbit-A488 Ab (ThermoFisher Cat No.: A11070) in green antibody diluent was added to sample sections and incubated at RT for 45 min. Slides were washed in TBS for 5 min at RT (repeat ×2). Sample sections were incubated with DAPI for 5 min at RT. Slides were washed in TBS for 5 min at RT (repeat ×2) and mounted with coverslip using Southernbiotech Fluoromount.

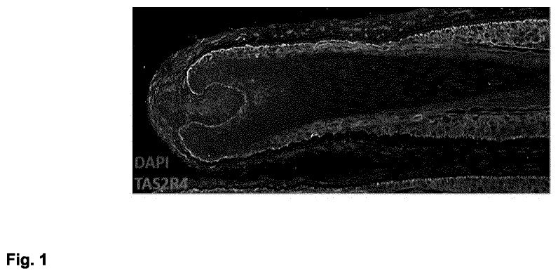

FIG. 1 shows the result of the immunofluorescence analysis of TAS2R4 expression in human anagen hair follicle according to the above protocol.

TAS2R4 expression was detected in the outer root sheath, hair matrix, dermal papilla, and connective tissue sheath.

2. Inactivation of TAS2R4 by RNAi Knockdown

For analyzing the effect of inactivating TAS2R4 by RNAi knock-down, hair follicles were cultured in William's E Medium (WEM) supplemented with 2 mM L-glutamine, 10 ng/mL hydrocortisone, 10 μg/mL insulin and 1% penicillin/streptomycin mix (WCM medium), and treated either with self-delivering scrambled oligos (Accell Non-targeting Control siRNA; Horizon Discovery Ltd, cat. D-001910-10-05) or with self-delivering siTAS2R4 (Accel siTAS2R4; Horizon Discovery Ltd, cat. E-013102-00-0005) according to the following procedure:

Day 0: Isolation of anagen hair follicles

Day 1: Change WCM media with self-delivery siRNA corresponding to experimental groups (Control group: 1 μM scrambled oligos, experimental group: 1 μM siTAS2R4)

Day 2: Rest

Day 3: Rest

Day 4: Collect 3 hair follicles/groups for qPCR (72h after siRNA delivery)

Day 5: Collect 5 hair follicles/groups for immunofluorescence (96h after siRNA delivery)

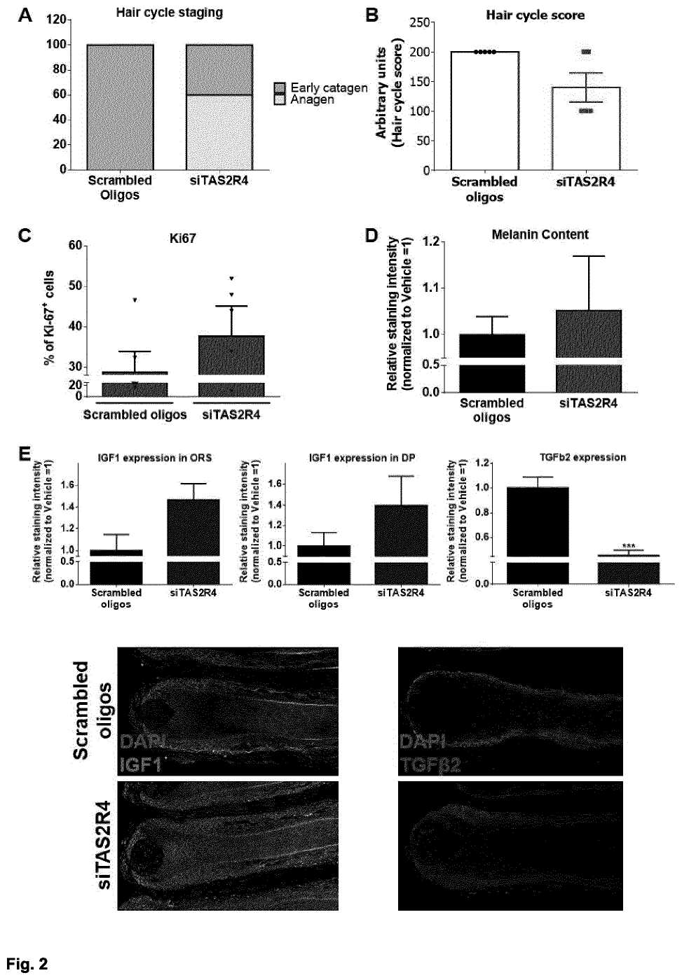

FIG. 2 shows the results of siRNA targeting TAS2R4 as mentioned above.

Inactivation of TAS2R4 by RNAi knock-down resulted in the anagen phase prolongation in the hair follicles ex vivo as shown with hair cycle staging and hair cycle score (HCS) (FIGS. 2A and 2B). HCS involves assigning an arbitrary unit for each stage of the hair cycle (Anagen VI=100; Early catagen=200; Mid-catagen=300; and Late catagen=400). After having classified each HF according to its hair cycle stage, the mean HCS was calculated. The closer the mean is to 100, the higher is the number of anagen VI HFs in a given group. The HCS provides a global readout parameter that looks at all HFs in a given experimental group and synthesizes them into a single number, which reflects how close the majority of HFs is to either anagen VI or catagen and also permits statistical analysis that it is not possible with hair cycle staging. Therefore, hair cycle staging and the HCS are independent readout parameters that complement each other.

TAS2R4 inactivation by RNAi knock-down was also associated with the increased number of proliferative Ki-67+ cells and the increased melanin content (FIGS. 2C and 2D).

In addition, the expression of a pro-anagenic growth factor IGF 1 and pro-catagenic growth factor TGFβ2 were increased and decreased respectively in response to the TAS2R4 inactivation by RNAi knock-down (FIG. 2E).

3. Inactivation of TAS2R4 with Nα,Nα-bis(carboxymethyl)-l-lysine (BCML)

For analyzing the effect of inactivating TAS2R4, by the inverse agonist Nα,Nα-bis(carboxymethyl)-l-lysine (BCML), hair follicles were cultured in WEM medium supplemented with 2 mM L-glutamine, 10 ng/mL hydrocortisone, 10 μg/mL insulin and 1% penicillin/streptomycin mix (WCM medium), and treated either with 120 μM BCML (Sigma-Aldrich, cat. 14580) diluted in WCM or vehicle (WCM medium) according to the following procedure:

Day 0: Isolation of anagen hair follicles

Day 1: Change media, application of the substance corresponding to experimental groups (control group: WCM, experimental group: 120 μM BCML)

Day 2: Rest

Day 3: Change media

Day 4: Rest

Day 5: Change media

Day 6: Rest

Day 7: Collection and freezing of hair follicles for analyses

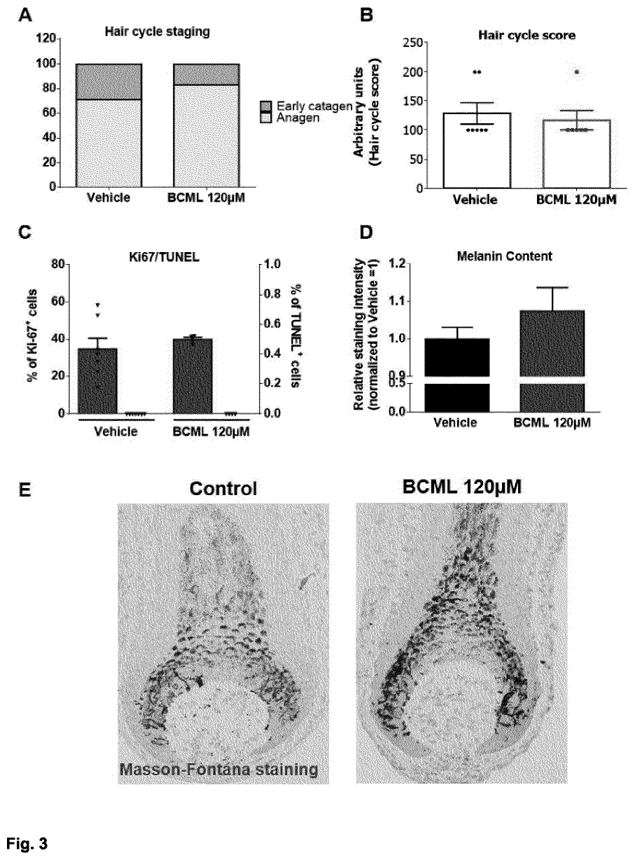

FIG. 3 shows the results of human hair follicles treated with 120 μM BCML.

Treatment of anagen hair follicles with the BCML increases the proportion of hair follicles in anagen stage (FIGS. 3A and 3B).

Further, treatment of human hair follicles with BCML increases hair matrix keratinocyte proliferation while does not affect apoptosis (FIG. 3C).

Finally, treatment of hair follicles with BCML increases pigmentation (FIGS. 3D and 3E).

4. Activation of TAS2R4 with Rebaudioside A

For analyzing the effect of activating TAS2R4 by the agonist Rebaudioside A, human anagen hair follicles from 2 donors were cultured in WEM medium supplemented with 2 mM L-glutamine, 10 ng/mL hydrocortisone, 10 μg/mL insulin and 1% penicillin/streptomycin mix (WCM medium), and treated either with 200 μM Rebaudioside A (Sigma-Aldrich, cat. 01432) diluted in DMSO or vehicle (0.5% DMSO in WCM medium) according to the following procedure:

Day 0: Isolation of anagen hair follicles

Day 1: Change WCM media, application of the substance corresponding to experimental groups (control group: 0.5% DMSO, experimental group: 200 μM Rebaudioside A)

Day 2: Rest

Day 3: Change media

Day 4: Rest

Day 5: Change media

Day 6: Collection and freezing of hair follicles for analyses

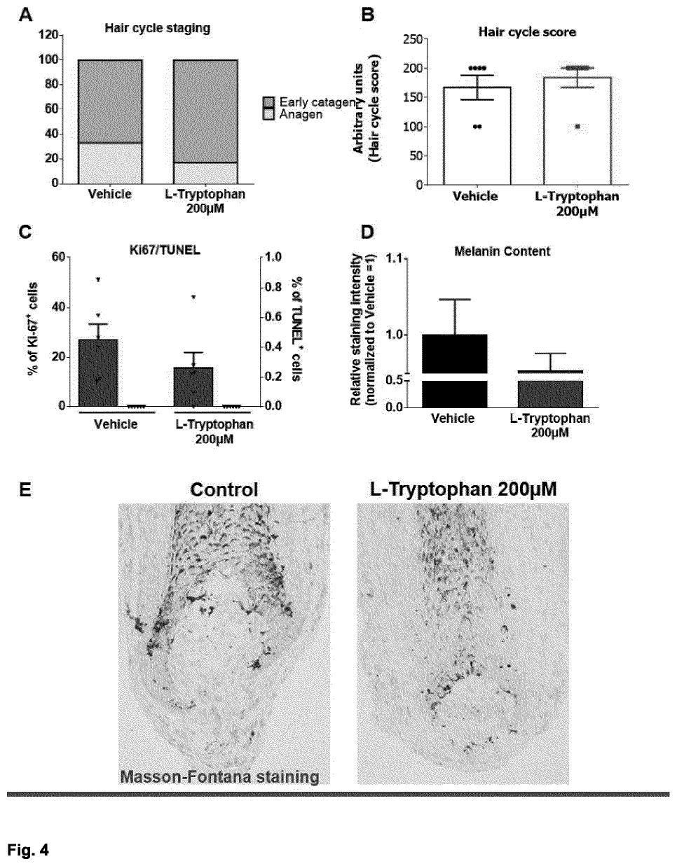

FIG. 4 shows the results of human hair follicles treated with 200 μM Rebaudioside A.

From the figures, it can be identified that activation of TAS2R4 by treatment of hair follicles with the Rebaudioside A increases the proportion of hair follicles in catagen stage (FIGS. 4A and 4B).

Further, treatment of human hair follicles with Rebaudioside A decreases hair matrix keratinocyte proliferation, and increases apoptosis (FIG. 4C).

Finally, Rebaudioside A treatment induces hypopigmentation, which is a sign of catagen induction (FIGS. 4D and 4E).

5. Activation of TAS2R4 with Rubusoside

For analyzing the effect of activating TAS2R4 by the agonist Rubusoside, hair follicles were cultured in WEM medium supplemented with 2 mM L-glutamine, 10 ng/mL hydrocortisone, 10 μg/mL insulin and 1% penicillin/streptomycin mix (WCM medium), and treated either with 50 μM Rubusoside (Sigma-Aldrich, cat. 62933) diluted in DMSO or vehicle (0.05% DMSO in WCM medium) according to the following procedure:

Day 0: Isolation of anagen hair follicles

Day 1: Change media, application of the substance corresponding to experimental groups (control group: 0.05% DMSO, experimental group: 50 μM Rubusoside)

Day 2: Rest

Day 3: Change media

Day 4: Rest

Day 5: Change media

Day 6: Collection and freezing of hair follicles for analyses

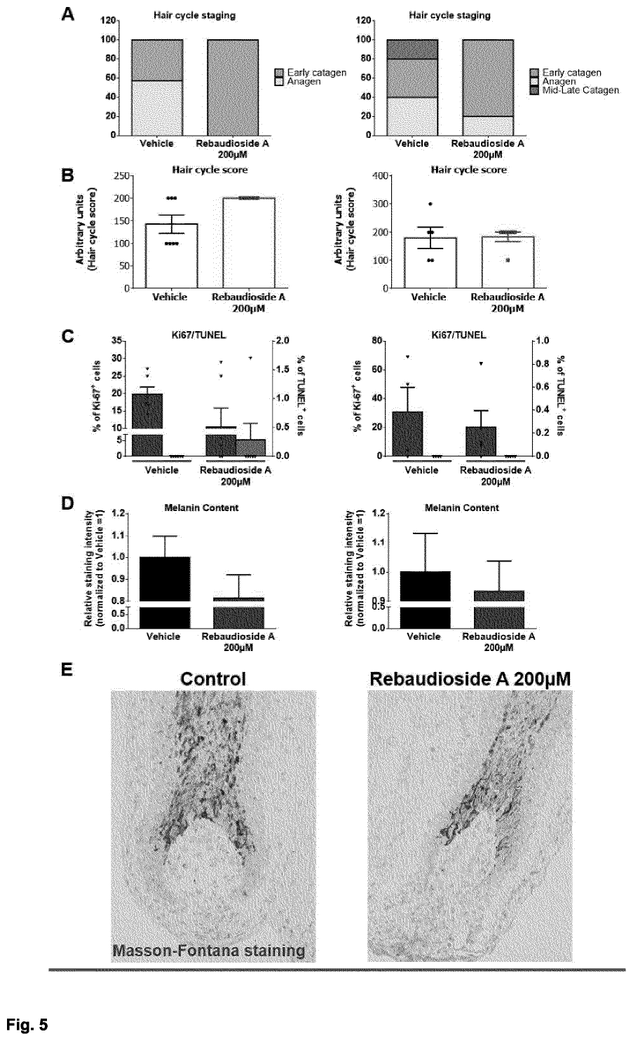

FIG. 5 shows the results of human hair follicles treated with 50 μM Rubusoside.

From the figures, it can be identified that activation of TAS2R4 by treatment of hair follicles with the Rubusoside increases the proportion of human hair follicles in catagen stage (FIGS. 5A and 5B).

6. Activation of TAS2R4 with Stevioside

To determine the effect of activating TAS2R4 by the agonist Stevioside, anagen hair follicles were cultured in WEM medium supplemented with 2 mM L-glutamine, 10 ng/mL hydrocortisone, 10 μg/mL insulin and 1% penicillin/streptomycin mix (WCM medium), and treated either with 200 μM Stevioside (Sigma-Aldrich, cat. 50956) diluted in DMSO or vehicle (0.2% DMSO in WCM medium) according to the following procedure:

Day 0: Isolation of anagen hair follicles

Day 1: Change media, application of the substance corresponding to experimental groups (control group: 0.2% DMSO, experimental group: 200 μM Stevioside)

Day 2: Rest

Day 3: Change media

Day 4: Rest

Day 5: Change media

Day 6: Rest

Day 7: Collection and freezing of hair follicles for analyses

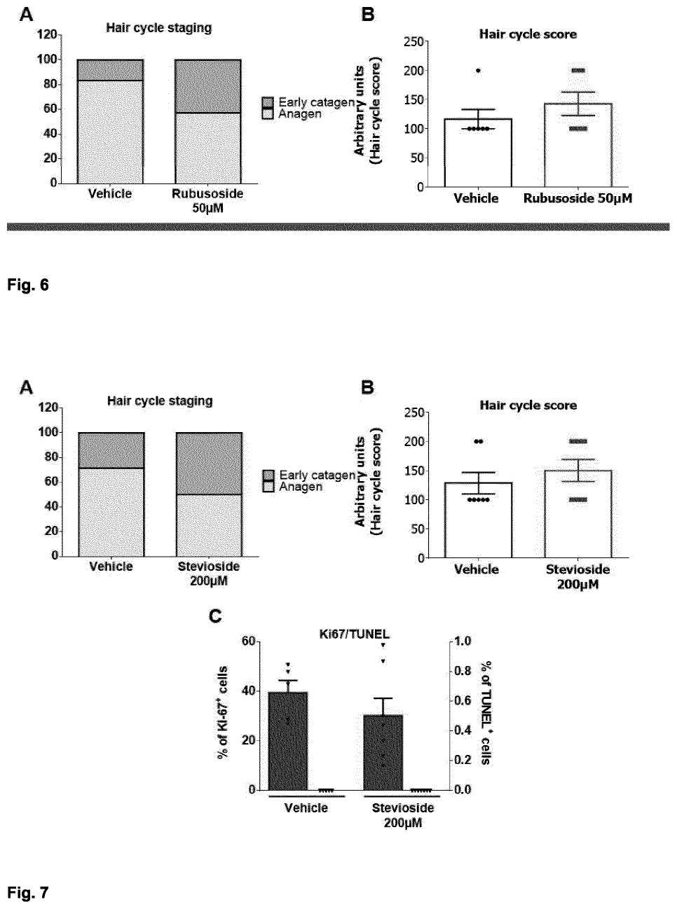

FIG. 6 shows the results of human hair follicles treated with 200 μM Stevioside.

From the figures, it can be identified that activation of TAS2R4 by treatment of anagen hair follicles with the Stevioside increases the proportion of hair follicles in catagen stage (FIGS. 6A and 6B).

Further, treatment of human hair follicles with Stevioside decreases hair matrix keratinocyte proliferation but does not affect apoptosis (FIG. 6C).

1. An active agent for use in the treatment of hair growth, wherein the active agent activates, enhances, inactivates, blocks or dampens the cellular response of taste receptor TAS2R4 or interferes with the expression of the receptor.

2. The active agent of claim 1, wherein the active agent is an agonist of the taste receptor TAS2R4 for use in the treatment of unwanted hair growth.

3. The active agent of claim 1, wherein the active agent is an antagonist/inverse agonist of the taste receptor TAS2R4 for use in the treatment of unwanted hair loss.

4. The active agent of claim 1, wherein the active agent is
a) any one of the TAS2R4 activating agonists amarogentin, arborescin, artemorin, azathioprine, chlorpheniramine, dapsone, D-camphor, denatonium benzoate, diphenidol, dulcoside A, (-)-epicatechin, leu-leu-leu, leu-trp, parthenolide, phe-trp, propylthiouracil, quassin, quinine, rebaudioside A, rebaudioside B, rebaudioside C, rubusoside, steviolbioside, stevioside, sucralose, taurocholic acid, trp-leu, trp-phe, trp-pro, trp-trp, trp-trp-trp, xanthoxin, yohimbine, or a combination thereof, or an aptamer binding to the TAS2R4 receptor and activating or enhancing the receptor to produce a cellular response: or
b) any one of the TAS2R4 inactivating antagonists/inverse agonists carboxymethyllysine (CML), Nα,Nα-bis(carboxymethyl)-1-lysine (BCML), glyoxal-derived lysine dimer (GOLD) and 4-(2,2,3-trimethylcyclopentyl) butanoic acid (GIV3727) or a combination thereof, or an aptamer binding to the TAS2R4 receptor and inactivating, blocking or dampening the receptor to produce a cellular response.

5. The active agent of claim 1, wherein the active agent is miRNA, siRNA or a ribozyme targeted to TAS2R4.

6. An active agent according to claim 1 for use as a cosmetic in the treatment of hair growth.

7. An active agent according to claim 1 for use as a medicament in the treatment of hair growth disorder.

8. An active agent for use as a medicament according to claim 7, wherein the hair growth disorder to be treated is effluvium, nonscarring alopecia, scarring alopecia, hypertrichosis or hirsutism.

9. A composition for use as a cosmetic or medicament in the treatment of hair growth, the composition comprising
at least one active agent according to claim 1; and
at least one auxiliary agent selected from among the group consisting of carriers, recipients, adjuvants, diluents, or disintegrants.

10. The composition of claim 9, wherein the auxiliary agent is selected from among the group consisting of liposomes, nanoparticles, carboxymethyl cellulose, hydroxyethyl cellulose, mineral oil, petrolatum, glycerin, polysorbate 80, hydroxyethyl starch, dextran, or polyethylene glycol.

11. The composition of claim 9, further comprising at least one other active agent being effective in the treatment of hair growth.

12. The composition of claim 9, wherein the composition is formulated in the form of an ointment, a lotion, a cream, a shampoo, a gel, a solution, a spray, a plaster or a sustained release plaster.

13. A Non-therapeutic method of hair growth regulation, comprising administering to a subject an effective amount of at least one active agent that activates, enhances, inactivates, blocks or dampens the cellular response of the taste receptor TAS2R4 or interferes with the expression of the receptor.

14. The non-therapeutic method of claim 13, wherein the active agent is:
a) any one of the TAS2R4 activating agonists, amarogentin, arborescin, artemorin, azathioprine,
chlorpheniramine, dapsone, D-camphor, denatonium benzoate, diphenidol, dulcoside A,
leu-leu-leu, leu-trp, parthenolide, phe-trp, propylthiouracil, quassin, quinine,
Rebaudioside A, Rebaudioside B, Rebaudioside C, Rubusoside, steviolbioside,
Stevioside, sucralose, taurocholic acid, trp-leu, trp-phe, tip-pro, trp-trp, trp-trp-trp,
xanthoxin, yohimbine, and (-)epicatechin or a combination thereof, or an aptamer binding to the TAS2R4 receptor and activating or enhancing the receptor to produce a cellular response; or
b) the TAS2R4 inactivating antagonists/inverse agonists carboxymethyllysine (CML), Nα,Nα-bis(carboxymethyl)-l-lysine (BCML), glyoxal-derived lysine dimer (GOLD) and 4-(2,2,3-trimethylcyclopentyl) butanoic acid (GIV3727), or an aptamer binding to the TAS2R4 receptor and inactivating, blocking or dampening the receptor to produce a cellular response.

15. The non-therapeutic method of claim 13, wherein the active agent is miRNA, siRNA or a ribozyme targeted to the TAS2R4 gene or targeted to the mRNA corresponding to the TAS2R4 gene.
